(19) United States
(12) Patent Application Publication
(10) Pub. No.: US 2006/0246481 A1
(43) Pub. Date: Nov. 2, 2006

(54) Methods of identifying cellular target molecules

(75) Inventors: Rosalynde J. Finch, Seattle, WA (US); David A. Basiji, Seattle, WA (US); William E. Ortyn, Bainbridge Island, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Amnis Corporation, Seattle, WA (US)

(21) Appl. No.: 11/364,858

(22) Filed: Feb. 27, 2006

Related U.S. Application Data
(63) Continuation of application No. 10/230,886, filed on Aug. 29, 2002, now abandoned.
(60) Provisional application No. 60/377,872, filed on May 3, 2002.
(60) Provisional application No. 60/334,479, filed on Nov. 30, 2001.

(51) Int. Cl. C12Q 1/68; C12N 1/08

(52) U.S. Cl. 435/6; 435/270

(57) ABSTRACT

The present invention provides methods of detecting and/or quantifying specific cellular target molecules in intact cells. The present invention further provides methods of processing an intact cell to facilitate in situ hybridization for use in flow cytometry.

### CROSS-REFERENCE TO RELATED APPLICATIONS

This application is a continuation application of U.S. patent application Ser. No. 10/230,886, filed Aug. 29, 2002, which claims the benefit under 35 U.S.C. § 119(e) of U.S. Provisional Patent Application No. 60/377,872, filed May 3, 2002, and U.S. Provisional Patent Application No. 60/334,479, filed Nov. 30, 2001, all of which are herein specifically incorporated by reference in their entireties.

### BACKGROUND OF THE INVENTION

1. Field of the Invention

The present invention relates to methods of detecting and/or quantifying specific cellular target molecules in intact cells. The present invention further relates to methods of processing an intact cell to facilitate in situ hybridization.

2. Description of the Related Art

In situ hybridization and immunohistochemistry are powerful means for detecting and/or quantifying a particular nucleic acid and/or protein, in a cell and/or cellular organelle. One particular advantage of in situ hybridization is its exceptional sensitivity, e.g., allowing a single copy of a gene to be detected in a cell. This widely employed technique relies on processing a cell and/or cellular organelle to allow a labeled probe (e.g., a nucleic acid having a specific nucleotide sequence) to permeate the cell and/or a cellular organelle membrane and then bind to a specific nucleic acid contained by the cell and/or cellular organelle. The binding of the labeled probe to the specific nucleic acid thereby enables the detection and/or quantification of the specific nucleic acid. Similarly, using immunohistochemistry, the binding of a labeled probe, i.e., a labeled antibody to a specific antigen of an intact fixed cell, enables the detection and/or quantification of that antigen.

In situ hybridization is generally performed on a substrate, such as a microscope slide and requires the fixing (e.g., with a polar organic solvent such as methanol) of the intact cells. However, this fixation step is not compatible with suspension hybridization due to the occurrence of substantial cell loss and cell clumping when transferring the fixed, alcohol-dehydrated cells into an aqueous saline-sodium citrate (SSC) buffer, used in the pre-hybridization step. Indeed, up to 90% of the cells fixed in 3:1 methanol:acetic acid are lost when they are transferred directly into 2×SSC, pelleted and then resuspended in the hybridization buffer. Moreover, the residual cells that do survive this transition then aggregate, making it difficult to clearly enumerate hybridization signals. Since flow cytometry works optimally when cells individually pass through the laser beam, this aggregation of the cells further hinders accurate analysis. Thus, whereas intact fixed cells can be readily mounted on a fixed substrate, heretofore, intact cells fixed with polar organic solvents have not been amenable for flow devices.

Therefore, methods for performing chromosomal in situ hybridization in suspension have heretofore employed isolated nuclei rather than morphologically intact cells (see, for example, Van den Engh and Trask, U.S. Pat. No. 4,770,992; Van Dekken et al. (1990) Cytometry 11:153-164). Isolated nuclei are fixed with a cross-linking agent such as dimethylsuberimidate (DMS) to stabilize the chromatin prior to denaturation of the chromosomal DNA and subsequent hybridization of a complementary nucleotide probe specific for the DNA sequence of interest. Alternatively the isolated nuclei can be fixed with ethanol to maintain their integrity during the denaturation and hybridization steps (Trask et al. (1988) Human Genetics 78:251-259). In either case, the nuclei are subsequently permeabilized with Triton X-100 to facilitate accessibility of the probe to chromosomal DNA. In another variation of the method, isolated nuclei are crosslinked with paraformaldehyde prior to the denaturation step, and then permeabilized with Tween-20 during the hybridization step (Arkesteijn et al. (1995) Cytometry 19:353-60).

Though performing in situ hybridization on isolated nuclei rather than on intact cells overcomes some of the aggregation problems associated with transitioning cells from 3:1 methanol:acetic acid into 2×SSC, the information obtained is more limited. Thus, when the cytoplasmic contents and cell membrane are removed, the analysis is limited to the components of the nucleus, such as chromatin and nuclear RNA. In many cases, it is desirable to analyze cytoplasmic nucleic acids, such as mRNA, and viral or bacterial nucleic acids in an infected cell. Furthermore, the proteins in the cytoplasm and on the cell surface that can be identified by labeled antibodies, for example, can only be preserved for analysis when the cytoplasm and cell membrane are maintained.

Methods for performing in situ hybridization on intact cells in suspension have been described by Timm et al. (1995) Cytometry 22:250-255, and Stewart and Timm, U.S. Pat. No. 5,436,144. In one case, oligonucleotide probes complementary to specific chromosome sequences are introduced into fixed cells and used to amplify the target sequence by the polymerase chain reaction (PCR). The cells are first fixed in formaldehyde, resuspended in 70% ethanol and then incubated in 1×SSC plus bovine serum albumin (BSA). The cells are then pelleted and resuspended in PCR buffer. After the PCR amplification of the target chromosomal sequence, a labeled probe complementary to the target sequence is hybridized to the amplified DNA. The bound, labeled probe is then detected by flow cytometry. Similar methods have been used to hybridize complementary probes to abundant cytoplasmic mRNAs without prior amplification (Bauman and Bentvelzen (1998) Cytometry 9:517-524; Timm and Carleton (1992) Biotechniques 12:362-367). Primed in situ hybridization (PRINS) has also been used in intact cells in suspension to detect specific mRNA sequences in the cytoplasm (Bains et al. (1993) Exp. Cell Res. 208:321-326), and to viral DNA and mRNA (Patterson et al. (1993) Science 260:976-979).

However, there are a number of disadvantages of amplifying target nucleic acids prior to performing in situ hybridization. First, it includes additional steps which add complexity to the process and require further time for performing the analysis. In addition, amplification steps make it difficult to quantify the original concentration of the target nucleic acid molecule. Moreover, non-specific nucleic acid amplification oftentimes leads to artifacts. Furthermore, subjecting the cell to repeated cycles of high temperature incubations during the amplification process can cause over-denaturization of the target nucleic acids and cell loss. Finally, the amplification process can result in the loss of spatial distinctions that are required for chromosomal enumeration.

Telomere fluorescent in situ hybridization (FISH) has been performed in suspension with whole cells (Rufer et al. (1998) Nat. Biotechnol. 16:743-747; Dragowska et al. (1998) Cytometry (Suppl.) 9:51; Hultdin et al. (1998) Nucleic Acids Res. 26:3651-3656.

Immunophenotyping has been used in combination with FISH for cells fixed to slides (Van den Berg et al. (1991) Lab. Invest 64:323-328; Weber-Matthieson et al. (1992) J. Histochem. Cytochem. 40:171-175; Weber-Mattieson et al. (1993) Cytogenetic Cell Genetics 63:123-125; Callet-Bauchu et al. (1997) Br. J. Haematol. 99:531-536).

### BRIEF SUMMARY OF THE INVENTION

Heretofore, however, no method has been described for direct in situ hybridization in intact cells in suspension without prior amplification of the target nucleic acid, unless the target) nucleic acid is an abundant cytoplasmic mRNA or a telomere sequence. Therefore, there is a need for new general methodology which would allow direct in situ hybridization in intact fixed cells in suspension without prior amplification. There is also a need for new general methodology which would allow direct immunochemical analysis in intact fixed cells in suspension. Furthermore, there is a need for new methodology that would allow the identification of specific cellular constituents (including cell surface antigens) of a single intact fixed cell in suspension, in conjunction with in situ hybridization.

In a first aspect, the invention features a method of identifying or quantifying a specific cellular target molecule of interest in an intact cell, comprising (a) treating the intact cell that contains or is suspected to contain a specific cellular target molecule with a solution that comprises a polar organic solvent, wherein the treated cell becomes fixed; (b) removing the fixed cell from the polar organic solvent; (c) rehydrating the cell in aqueous buffer; (d) exposing the rehydrated cell to a hybridization buffer; (e) contacting the cell of step (d) with a probe able to hybridize to the specific cellular target molecule of interest; and (f) detecting the hybridized target molecule of interest. The method of the invention can be performed with an intact cell immobilized on a subtrate, e.g., solid support, microscope slide, microtiter well, or membrane, or an intact cell in suspension.

In one such embodiment, the polar organic solvent is a short-chain alcohol. In specific embodiments of the invention, the polar organic solvent is methanol, ethanol, or acetone.

In one embodiment the solution further comprises an aqueous component. In a preferred embodiment, the solution is 3:1 methanol:acetic acid.

In certain embodiments, the intact cell is hypotonically swelled prior to being fixed. In a specific embodiment, the cell is swelled in a hypotonic salt solution, such as 0.56% KCl as exemplified below, for about 10 to 20 minutes at a temperature of about 25° C. to 37° C.

In one embodiment, the fixed cell is removed from the solution in step (b) by centrifugation, although any means of isolating a cell known to the art may be used. In a specific embodiment, the rehydrating step is performed by resuspending the fixed cell in 1× saline-sodium citrate (SSC)+0.05% bovine serum albumin (BSA) at 25° C. for about 20 minutes to 1 hour or longer. The rehydrated cell is then placed in an aqueous buffer that is similar to the hybridization buffer. In Example 1 below, a rehydrated cell is placed in 2×SSC+0.05% BSA for about 5 to 20 minutes prior to being placed into the hybridization buffer of 2×SSC, 50% formamide, 10% dextran sulfate. The rehydrated cell is then removed from the aqueous buffer and exposed to a hybridization buffer. In a specific embodiment, the hybridization buffer is 2×SSC, with 50% formamide, and 10% dextran sulfate. In another embodiment, the rehydration step is conducted in a step-wise process in which the cell is placed in 1×SSC buffer followed by 2×SSC buffer.

In one embodiment, the resuspended cell is contacted with a probe capable of entering the cell and binding or hybridizing to the specific cellular target molecule of interest. Alternatively, the probe may bind to a specific cell surface antigen, though cell surface antigens are preferably labeled prior to, rather than following the fixation of the cell. In any case, the probe facilitates the identification of the specific cellular target molecule by making it detectable. The specific cellular target molecule of the fixed cell is then detected and/or quantified allowing the specific cellular target molecule to be identified and/or detected and/or quantified.

In one embodiment, the probe is labeled. In another embodiment, the method further comprises contacting the probe to a binding partner that contains a label. In a specific embodiment, the label is a luminescent label, a light absorbing label, a readioactive label, or a light scattering label. The methods of the present invention include any and/or all of the necessary wash steps to remove excess probe and/or label that is not bound either directly or indirectly to the cell and/or a constituent thereof.

In one embodiment the specific cellular target molecule of interest is a specific protein. In a more specific embodiment, the protein is a cytokine, such as tumor necrosis factor α or interferon γ, or a hemoglobin chain, e.g., a fetal hemoglobin chain or an adult hemoglobin chain. In another embodiment, the specific cellular target molecule of interest is a nucleic acid. In further specific embodiments, the specific cellular target molecule of interest is a lipid, phospholipid, glycolipid, or a carbohydrate.

In one embodiment, the probe is a nucleic acid. In a specific embodiment, both the probe and the specific cellular target molecule are nucleic acids. In another embodiment the probe is an antibody. In a more specific embodiment of this type, the specific cellular target molecule is a protein and the probe is an antibody.

In a second aspect, the invention features a method of detecting a specific cellular target molecule of interest in an intact cell, comprising (a) treating the intact cell that contains or is suspected to contain a specific cellular target molecule with a solution that comprises a polar organic solvent, wherein the treated cell becomes fixed; (b) removing the fixed cell from the polar organic solvent; (c) rehydrating the cell in aqueous buffer; (d) exposing the rehydrated cell to a hybridization buffer; (e) contacting the cell of step (d) with a probe able to hybridize to the specific cellular target molecule of interest; and (f) detecting the hybridized target molecule of interest. The method of the invention can be performed with an intact cell immobilized on a subtrate, e.g., solid support, microscope slide, microtiter well, or membrane, or an intact cell in suspension.

In a third aspect, the invention features methods of identifying, detecting, or quantifying a specific cell surface antigen on a cell that has or is suspected to have the specific cell surface antigen prior to performing in situ hybridization. One such method comprises contacting (e.g., incubating) an intact cell with an antibody that binds to the specific cell surface antigen such that when the cell comprises the specific cell surface antigen, the antibody binds to the specific cell surface antigen of the cell. The cell is then treated with a solution comprising a polar organic solvent. Such treatment results in the cell becoming fixed. The fixed cell is then isolated (removed) from the solution and the isolated cell is incubated in an aqueous buffer. The resulting rehydrated cell is then contacted, e.g., exposed or placed in a hybridization buffer, and contacted with a complementary probe able to hybridize to the specific cellular target molecule of interest (i.e., a chromosome or gene sequence). Following in situ hybridization, the antibody bound to the cell surface antigen is detected and/or quantified. The specific cell surface antigen is then identified/detected and/or quantified if the antibody is detected and/or quantified. In a particular embodiment the antibody comprises a label. In an alternative embodiment, the method further comprises contacting the antibody with a binding partner that comprises a label following placing the cell in the hybridization buffer. In this case, the detecting of the antibody is performed by detecting the label of the labeled binding partner of the antibody. In specific embodiments, the cell is immobilized on a substrate, or in suspension.

In a fourth aspect, the invention also provides specific methods of identifying/detecting and/or quantifying a specific cellular target molecule in an intracellular location in intact cell with an antibody, prior to in situ hybridization. One such method comprises treating the cell with a solution comprising a polar organic solvent, such that the cell becomes fixed. The fixed cell is then isolated (removed) from the solution and incubated in an aqueous buffer. The resulting rehydrated cell is then isolated from the aqueous buffer and an antibody that recognizes an intracellular site of the specific intracellular target molecule is contacted with (e.g., added to) the cell to allow the antibody to enter the cell and bind to the specific intracellular target molecule. The antibody thereby facilitates the identification of the specific cellular target molecule by making it detectable. The isolated rehydrated cell is then placed in a hybridization buffer and contacted with a complementary probe to a specific cellular target molecule (i.e., chromosome or gene sequence). Following in situ hybridization, the antibody bound to the specific intracellular target molecule of the resuspended cell is identified/detected and/or quantified thereby allowing the specific cellular target molecule to be identified/detected and/or quantified.

In a particular embodiment, the antibody comprises a label. In an alternative embodiment, the method further comprises contacting the antibody with a binding partner that comprises a label following exposure of the cell to hybridization buffer. In this case, the detecting of the antibody is performed by detecting the label of the labeled binding partner of the antibody. In specific embodiments, the intact cell is immobilized on a substrate or in suspension.

The invention further features a method of detecting a target molecule in an intact cell involving treating a cell with an aldehyde fixative, followed by a limited heat treatment step prior to in situ hybridization in suspension. Cross-linking fixatives, such as aldehydes, do not cause cell aggregation, so that the aldehyde-fixed cells may be analyzed by flow cytometry, but aldehyde fixation normally renders the chromosomal DNA inaccessible to hybridization with nucleic acid probes. However, the use of a limited fixation step in conjunction with a heat treatment step to reverse the aldehyde-induced crosslinking between chromosomal DNA and nuclear proteins was found to render the chromosomal DNA accessible to probes.

Accordingly, in a fifth aspect, the invention features a method of identifying, detecting, or quantifying a specific cellular target molecule of interest in an intact cell, comprising (a) treating the intact cell that contains or is suspected to contain a specific cellular target molecule with an aldehyde fixative, wherein the treated cell becomes fixed; (b) exposing the aldehyde-treated cell to a heat treatment; (c) placing the cell in a hybridization buffer; (d) contacting the cell of step (c) with a probe able to hybridize to the specific cellular target molecule of interest; and (e) detecting the hybridized target molecule of interest.

The intact cell of the method of the invention may be immobilized on a substrate or in suspension. In specific embodiments, the aldehyde fixative is selected from the group consisting of formaldehyde, paraformaldehyde, and glutaraldehyde.

In another embodiment, the heat treatment of step (b) is conducted at a temperature between about 50° C.-70° C. In a more specific embodiment, the temperature is about 65° C. The length of time required to reverse aldehyde-induced crosslinking depends on the type and percentage of aldehyde used for fixation, and the length of the fixation time. In one embodiment, the heat treatment of step (b) is conducted between 30 min to 5 hours; in a more specific embodiment, between 1-4 hours.

In one embodiment, when it is desirable to perform in situ hybridization on slides, the cells treated with aldehyde and heat can be resuspended in methanol and dropped on slides for conventional in situ hybridization.

In one embodiment, the isolated cell is contacted with a probe capable of entering the cell and binding or hybridizing to the specific cellular target molecule of interest. Alternatively, the probe may bind to a specific cell surface antigen, although cell surface antigens may be labeled prior to, rather than following the fixation of the cell. In any case, the probe facilitates the identification of the specific cellular target molecule by making it detectable. The specific cellular target molecule of the fixed cell is then detected and/or quantified allowing the specific cellular target molecule to be identified and/or detected and/or quantified.

In one embodiment, the probe is labeled. In another embodiment, the method further comprises contacting the probe to a binding partner that contains a label. In specific embodiments, the label is a luminiscent, light-absorbing, radioactive, or light-scattering label. The methods of the present invention include any and/or all of the necessary wash steps to remove excess probe and/or label that is not bound either directly or indirectly to the cell and/or a constituent thereof.

In one embodiment the specific cellular target molecule of interest is a specific protein. In a more specific embodiment, the protein is a cytokine, such as tumor necrosis factor α or interferon γ, or a hemoglobin chain, e.g., a fetal hemoglobin chain or an adult hemoglobin chain. In another embodiment, the specific cellular target molecule of interest is a nucleic acid. In further specific embodiments, the specific cellular target molecule of interest is a lipid, phospholipid, glycolipid, or a carbohydrate.

In one embodiment, the probe is a nucleic acid. In a specific embodiment, both the probe and the specific cellular target molecule are nucleic acids. In another embodiment the probe is an antibody. In a more specific embodiment of this type, the specific cellular target molecule is a protein and the probe is an antibody.

In a sixth aspect, the invention features methods of identifying, detecting, or quantifying a specific cell surface antigen on a cell that has or is suspected to have the specific cell surface antigen. One such method comprises contacting (e.g., incubating) a cell with an antibody that binds to the specific cell surface antigen such that when the cell comprises the specific cell surface antigen, the antibody binds to the specific cell surface antigen of the cell. The cell is then treated with an aldehyde fixative, resulting in the cell becoming fixed, followed by a limited heat treatment under defined conditions. The heat treated cell is placed in a hybridization buffer, and the cellular target molecule (i.e., chromosome or gene sequence) is contacted with a complementary probe. The specific cell surface antigen is then identified, detected and/or quantified if the antibody is detected and/or quantified. This method of the invention can be used with an intact cell that is immobilized on a substrate, or that is in suspension. In a particular embodiment the antibody comprises a label. In an alternative embodiment, the method further comprises contacting the antibody with a binding partner that comprises a label following in situ hybridization. In this case, the detecting of the antibody is performed by detecting the label of the labeled binding partner of the antibody.

The present invention also provides specific methods of identifying, detecting and/or quantifying a specific cellular target molecule in an intact cell with an antibody that recognizes a specific cellular target molecule in an intracellular location. One such method comprises treating the cell with an aldehyde fixative, followed by a permeabilization step. The fixed and permeabilized cell is then contacted with an antibody that recognizes the specific intracellular target molecule under conditions in which allow the antibody to enter the cell and bind to the specific cellular target molecule. The antibody thereby facilitates the identification of the specific intracellular target molecule by making it detectable. The antibody-labeled cell is then treated with methanol to fix the antibody to the intracellular target molecule. The cell is then subjected to a heat treatment to reverse crosslinking between chromosomal DNA and nuclear proteins. The isolated cell is then placed in a hybridization buffer and subjected to in situ hybridization. Following in situ hybridization, the antibody bound to the specific cellular target molecule of the resuspended cell is identified/detected and/or quantified thereby allowing the specific intracellular target molecule to be identified/detected and/or quantified. This method of the invention may be used with an intact cell immobilized on a substrate or in suspension.

In a particular embodiment the antibody comprises a label. In an alternative embodiment, the method further comprises contacting the antibody with a binding partner that comprises a label following the resuspending of the cell in the hybridization buffer. In this case, the detecting of the antibody is performed by detecting the label of the labeled binding partner of the antibody.

The present invention also provides kits for performing the various methods of the present invention. In a particular embodiment, the kits comprise the reagents needed to perform one or more of the methods of the present invention. In a preferred embodiment the kit also includes a protocol, e.g., directions, for using the reagents to identify and/or detect and/or quantify a specific cellular target molecule in an intact cell. In another preferred embodiment, the kit also includes a probe that can bind to a specific cellular target molecule in an intact cell. In a particular embodiment of this type, the probe contains a label.

In one particular embodiment, the kit is designed to be used with a directly-labeled probe in an in situ hybrididization (ISH) assay of a cell in suspension. In another embodiment, the kit is designed to be used with a directly-labeled probe in an ISH assay of a cell on a solid substrate. In yet another embodiment the kit is designed to be used with an indirectly-labeled probe in an ISH assay of a cell in suspension. In still another embodiment the kit is designed to be used with an indirectly-labeled probe in an ISH assay of a cell on a solid substrate. In a preferred embodiment, the kit is for performing FISH and the label is a fluorophore.

In yet another embodiment, the kit is designed to be used with an antibody to detect a specific cell surface antigen in an intact cell in solution. In still another embodiment, the kit is designed to be used with an antibody to detect a specific cell surface antigen on a solid substrate. In yet another embodiment, the kit is designed to be used with an antibody to detect a specific intracellular target molecule in an intact cell in solution. In still another embodiment, the kit is designed to be used with an antibody to detect an intracellular site of a specific intracellular target molecule on a solid substrate. In a preferred embodiment the antibody is a labeled antibody. More preferably, the label of the labeled antibody is a fluorophore.

In a particular embodiment, the identifying, detecting, or quantifying is performed in conjunction with flow cytometry. In a specific embodiment, the specific cellular target molecule is detected with imaging flow cytometry. In another specific embodiment, the specific cellular target molecule is detected by time delayed and integration (TDI) imaging flow cytometry. In the most preferred embodiment, the TDI imaging flow cytometer is one that is described in U.S. Pat. No. 6,211,955 and/or U.S. Pat. No. 6,249,341, the contents of which are herein specifically incorporated by reference in their entireties. In another embodiment, the specific cellular target molecule is detected by conventional flow cytometry, light microscopy, or fluorescence microscopy.

Other objects and advantages will become apparent from a review of the ensuing detailed description.

### DETAILED DESCRIPTION OF THE INVENTION

Before the present methods and compositions are described, it is to be understood that this invention is not limited to particular methods, compositions, and experimental conditions described, as such methods and compounds may vary. It is also to be understood that the terminology used herein is for the purpose of describing particular embodiments only, and is not intended to be limiting, since the scope of the present invention will be limited only to the appended claims.

As used in this specification and the appended claims, the singular forms “a”, “an”, and “the” include plural references unless the context clearly dictates otherwise. Thus for example, references to “a cell” or “the method” includes one or more cells or methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are specifically incorporated by reference.

General Description

The present invention provides novel methods of performing in situ hybridization and immunohistochemistry. A preferred aspect of the invention includes a processing step that allows cells that have been fixed to undergo in situ hybridization or immunohistochemistry in a cell suspension, without cell clumping and/or without a significant loss of cells. In one embodiment of a method of the invention, a processing step is performed by rehydrating the fixed cells prior to the denaturation and hybridization steps of conventional in situ hybridization and immunohistochemical analyses.

Thus, in one embodiment, the present invention provides a method of identifying a known target nucleic acid sequence contained in a morphologically intact cell under conditions in which the cell is maintained in a suspension rather than fixed on a surface, thereby allowing the detection of the presence of the target nucleic acid sequence in the cell.

A particular embodiment of the present invention uses a polar organic solvent (i.e., methanol, ethanol, or acetone) or a mixture of a polar organic solvent and an aqueous solution, such as a methanol:acetic acid solution to fix intact cells and render them permeable to nucleic acid and/or antibody probes. The cells prepared can be used either for conventional in situ hybridization or immunochemistry on slides for example, or they can be further processed for in situ hybridization or immunohistochemical analysis in suspension.

In conventional in situ hybridization performed on a solid substrate, the fixed cells are dehydrated by a series of stepwise increases in concentrations of ethanol until the cells are in 100% ethanol. The cells are then washed in 2×SSC prior to hybridization in a 2×SSC/50% formamide buffer containing the nucleic acid probe.

The present invention provides a novel method of performing in situ hybridization in suspension. In one embodiment, the cells are rehydrated stepwise in 1×SSC plus BSA, then 2×SSC plus BSA followed by hybridization in 2×SSC/50% formamide buffer. The stepwise rehydration provided by the present invention eliminates the cell loss and clumping incurred when methanol, ethanol, or acetone dehydrated cells are transferred directly into an aqueous solution such as 2×SSC. Thus, the present invention provides a method of maintaining fixed cells in a single cell suspension that is compatible with flow cytometry.

In a second method of the invention of performing in situ hybridization in suspension, the cells are exposed to an aldehyde fixative followed by heat treatment prior to resuspension for in situ hybridization. This method of the invention does not require a rehydration step and does not result in cell aggregation. The method of the invention encompassing aldehyde fixation and a limited heat treatment may also be used with cell surface antigen labeling and intracellular antigen labeling protocols.

Definitions

As used herein a “specific cellular target molecule” is a specific cellular constituent that is amenable to being detected either directly or indirectly through its binding to a binding partner (e.g., a ligand to its receptor, an antibody, or a complementary nucleic acid). Specific cellular target molecules include intracellular constituents and cell membrane constituents such as a cell surface antigen. In a particular embodiment, a specific cellular target molecule is a specific protein, e.g., interferon γ, tumor necrosis factor α (TNFα) or a hemoglobin chain. In a preferred embodiment, a specific cellular target molecule is a specific nucleic acid such as one that comprises a nucleotide sequence that is unique to a particular gene or chromosome, e.g., a nucleotide sequence found only in the gene encoding fetal hemoglobin.

As used herein, an “antibody that recognizes an intracellular site of a specific cellular target molecule” is an antibody that recognizes an antigenic site of the specific cellular target molecule that is inside the cell, as opposed to being in and/or on the outer membrane of cell.

As used herein a cell that has been dehydrated, (e.g., via incubation with a polar organic solvent) is “rehydrated” when it is placed into an aqueous solution and can be easily resuspended in an aqueous buffer used in hybridization and post-hybridization washes. Generally, rehydration buffers also include one or more proteins such as bovine serum albumin (BSA) or fetal bovine serum (FBS), to help reduce the cells sticking to the tubes.

As used herein, a “hybridization buffer” is a solution that contains a buffering agent and is suitable for the binding of a probe to its corresponding specific cellular target molecule in an intact cell, see Examples below. Preferably, a hybridization buffer also comprises a blocking agent to reduce non-specific binding. In addition, a hybridization buffer may contain a chaotropic agent, e.g., to lower the melting temperature (Tm) for hybridization. A hybridization buffer may also contain a target-specific probe.

In one method of the invention, polar organic solvents (e.g., acetone or short chain alcohols such as methanol and ethanol) and mixtures of polar organic solvents with aqueous solutions (e.g., 3:1 methanol:acetic acid) are used; in another embodiment, aldehyde fixatives are used, followed by a heat treatment to reverse crosslinking of chromosomal DNA and nuclear proteins in order to allow access to chromosomal DNA. Further, other fixatives can be employed for specific uses including certain salt solutions such as sodium sulfate or cesium acetate. Preferably, a fixing solution fixes the cellular constituents of a cell through a precipitating action. More preferably, the cellular morphology of a fixed cell is maintained, the antigenicity of the cell cellular constituents is maintained, the nucleic acids are retained in the appropriate location of the cell and the nucleic acids maintain their ability to hybridize.

Polar organic solvents such as methanol, ethanol, and acetone are believed to denature proteins by removing water molecules that are bound to the hydrophilic residues of the protein. Polar organic solvents also can remove some of the membrane and structural lipids making the cells more permeable, including allowing antibody molecules to enter the cell. Intracellular as well as cell membrane antigens are usually well preserved in polar organic fixatives. However, treating cells with methanol, ethanol and/or acetone results in subsequent cell clumping in aqueous buffers, unless cells are gradually rehydrated in a stepwise process.

Aldehydes are thought to fix cells by denaturation and chemical modification of proteins, i.e., by covalent reaction with free amino groups of, e.g., lysine residues. The fixation frequently alters peptide chain antigens of intracellular proteins while the glycoantigens of the cell membrane glycocalix remain largely unaffected. Cells become rigid because protein cross linkings occur and consequently the cells suspend well. However, the cell membranes of intact cells remain relatively impenetrable to larger molecules such as antibodies unless permeabilized with detergents such as Tween 20, Triton X-100, or saponin. Furthermore, chromosomal DNA is inaccessible to hybridization with nucleic acid probes unless treated with heat to reverse aldehyde-induce crosslinking between chromosomal DNA and nuclear proteins.

Although not essential for the methodology disclosed herein, certain types of cells (e.g., amniocytes) are preferably hypotonically swelled prior to the fixation process. Hypotonically swelling a cell enlarges the cell nucleus allowing, e.g., the label in an in situ hybridization to be more readily discerned. A cell is hypotonically swelled when it is placed into a hypotonic saline solution, thereby being subjected to hypotonic swelling.

Source of Cells

Cells for the present invention can be obtained from any source and/or from any organism, or tissue from any organism, and can be living or dead. In a preferred embodiment the cells are human cells. Examples of appropriate sources include, cells obtained from the placenta, skin, or bone marrow, cells from any body fluids including blood, urine, sputum, amniotic fluid, effusions, and breast milk, tissue biopsies such as kidney, liver or brain biopsies, tumor biopsies, and cells obtained from lavage (e.g., ductal, alveolar, or peritoneal). Cells derived from any tissue that have been adapted for tissue culture also can be used.

Throughout the procedures disclosed herein, a need arises to transfer the cells employed, including fixed cells, from one solution to another. This transfer can initially entail the isolation/removal of the cells from a particular solution prior to placing them into a second solution. The cells employed by the present invention including fixed cells, can be isolated from a solution by any number of means including by centrifugation as exemplified below, cell sorting (e.g., FACS), lyophilization, and by filtration (e.g., with a membrane that retains the cells but not the solvent).

In addition, a fixed cell can be isolated from a fixation solution for example, by magnetic separation. Thus, a specific cellular target molecule such as a cell surface antigen or a cellular target molecule with an intracellular antigenic site, can be bound by an antibody that is conjugated to a magnetic bead (Miltenyi Biotec:Bergisch Gladbach, Germany, or Dynal: Oslo, Norway). Antibody binding to cell surface molecules can take place prior to fixation or following fixation, depending on the antibody:antigen interaction. Intracellular antibody binding takes place following fixation and permeabilization of the cell, and preferably uses Miltenyi beads because of their smaller size. Certain cell types that have phagocytic properties can also engulf magnetic particles or beads prior to fixation. The magnetically-labeled cells are then separated from the solution using a strong magnet, and can be washed and resuspended in the aqueous hybridization buffer without loss of the magnetic label.

Microscopy and Flow Cytometry

Cells on a fixed substrate can be detected by a brightfield microscope which is particularly useful for enzymatic stains (e.g., peroxidase). Alternatively, a fluorescence microscope can be employed for fluorescent labels. In addition, digital imaging can be used and provides both superior resolution and the ability to obtain more accurate quantitative determinations.

Individual cells in solution can be monitored by flow cytometry (e.g., a Coulter Profile II flow cytometer, or a FACS-Vantage or Calibur cytometer, Becton Dickinson). In standard flow cytometry the cells pass in a single file through a light source which is preferably a laser beam. A labeled probe bound to a specific cellular target molecule can absorb the light (ultraviolet, visible and/or infrared) and the effect of the light on the labeled probe can then be monitored, e.g., as simple absorbance and/or as subsequent fluorescence emission and/or as chemiluminescence/bioluminescence. For example, a photomultiplier can be aligned with the light source when the absorbance of the labeled probe is being monitored so as to allow the determination of the absorbance due to the labeled probe. In a particular embodiment the photomultiplier is perpendicular to the light source when the fluorescence of the labeled probe is being monitored, so as to allow the emission due to the labeled probe to be detected with minimal interference from the initial light source.

Recently, an important advance in flow cytometry has been disclosed in the imaging and analysis of cells. This methodology employs an optical dispersion system in combination with a time delay and integration detector that produces an output signal in response to the images of cells that are directed on the time delay and integration detector (U.S. Pat. Nos. 6,211,955 and 6,249,341). The methods of the present invention are compatible for use with this new technology, as well as with the more conventional flow cytometry and microscopy methodology.

Probes

The probes of the present invention include binding partners for any specific cellular target molecule. Particular probes include nucleotide probes (e.g., DNA, RNA or peptide nucleic acids, i.e., PNA), antibodies (polyclonal or monoclonal), and specific ligands including protein ligands. The probes can be directly labeled or can be indirectly-labeled. For nucleic acid probes, the size of the probe can be adjusted to detect a single mismatch (e.g., 12-50 bases) though much larger probes also can be employed. Once a specific cellular target molecule is selected, the skilled artisan can readily prepare an appropriate probe, be it an antibody to a protein or a nucleic acid probe for a particular nucleic acid.

Labels

The probes of the present invention including proteins (antibodies), and nucleic acids can all be labeled. Suitable labels include enzymes, fluorophores such as fluorescein, or a derivative thereof, e.g., fluorescein isothiocyanate (FITC), phycoerythrin (PE), Texas red (TR), Cy Dye, rhodamine, alexa dyes, free or chelated lanthanide series salts, especially Eu³⁺, chromophores, radioisotopes, chelating agents, dyes, colloidal gold, latex particles, ligands (e.g., biotin), and chemiluminescent agents.

In the instance where the label is an enzyme, detection may be accomplished by any of the presently utilized colorimetric, spectrophotometric, fluorospectrophotometric, amperometric or gasometric techniques known in the art.

Direct labels are one example of labels that can be used according to the present invention. A direct label has been defined as an entity, which in its natural state, is readily visible, either to the naked eye, or with the aid of an optical filter and/or applied stimulation, e.g. ultraviolet light to promote fluorescence or by infrared spectroscopy. Among examples of colored labels, which can be used according to the present invention, include metallic sol particles, for example, gold sol particles such as those described by Leuvering (U.S. Pat. No. 4,313,734); dye sole particles such as described by Gribnau et al. (U.S. Pat. No. 4,373,932) and May et al. (WO 88/08534); dyed latex such as described by May, supra, Snyder (EP-A 0 280 559 and 0 281 327); or dyes encapsulated in liposomes as described by Campbell et al. (U.S. Pat. No. 4,703,017). Other direct labels include a radionucleotide, a fluorescent moiety or a luminescent moiety. In addition to these direct labeling devices, indirect labels comprising enzymes can also be used according to the present invention. Various types of enzyme linked immunoassays are well known in the art, for example, alkaline phosphatase and horseradish peroxidase, lysozyme, glucose-6-phosphate dehydrogenase, lactate dehydrogenase, urease. These and others have been discussed in detail by Engvall in Enzyme Immunoassay ELISA and EMIT (1980) Methods in Enzymology 70:419-439, and in U.S. Pat. No. 4,857,453. In addition, cellular proteins (including membrane proteins) can be modified to contain a marker protein such as green fluorescent protein as described for example in U.S. Pat. No. 5,625,048, WO 97/26333, and WO 99/64592, the contents of which are hereby incorporated by reference in their entireties.

In the instance where a radioactive label, such as the isotopes ³H, ¹⁴C, ³²P, ³⁵S, ⁵¹Cr, ⁵⁷Co, ⁵⁸Co, ⁵⁹Fe, ⁹⁰Y, ¹²⁵I, ¹³¹I, and ¹⁸⁶Re can be used, known currently available counting procedures may be utilized. Proteins, including antibodies, can be labeled by metabolic labeling. Metabolic labeling occurs during in vitro incubation of the cells that express the protein in the presence of culture medium supplemented with a metabolic label, such as [³⁵S]-methionine or [³²P]-orthophosphate. In addition to metabolic (or biosynthetic) labeling with [³⁵S]-methionine, the invention further contemplates labeling with [¹⁴C]-amino acids and [³H]-amino acids (with the tritium substituted at non-labile positions).

Other labels for use in the invention include magnetic beads or magnetic resonance imaging labels. In another embodiment, a phosphorylation site can be created on an antibody of the invention for labeling with ³²P, e.g., as described in European Patent No. 0372707, U.S. Pat. No. 5,459,240, or U.S. Pat. No. 5,986,061.

Kits

The present invention also provides kits for performing the methods of the present invention. Generally, such kits will comprise the reagents needed to perform the methods of the present invention. In a preferred embodiment the kits will also include a protocol, e.g., directions, to use the reagents. Preferably, the kits provide the reagents in concentrations that can be readily used and which have been optimized with respect to the method to be followed. In a preferred embodiment, a probe that binds and/or reacts with a specific cellular target molecule in an intact cell is included. More preferably, the probe is a labeled probe.

A kit of the present invention can be for general use, i.e., employed in more than one type of assay, i.e., capable of examining cells on a solid substrate or in solution. Alternatively, the present invention provides special kits that employ specific reagents such as those detailed in the Examples below, for specific applications.

One kit of the present invention comprises (i) a fixing solution such as 3:1 methanol:acetic acid, (ii) a rehydrating buffer such as 1×SSC+0.05% BSA, and (iii) a hybridization buffer, such as 2×SSC, 50% formamide, 10% dextran sulfate.

In related kit, a pre-hybridization buffer is also supplied, e.g., 2×SSC+0.05% BSA. In still another kit, a washing solution such as phosphate-buffered saline (PBS) is also included. In addition, a hypotonic solution to hypotonically swell the cells, such as 0.56% KCl can also be included. Nuclear counterstains such as DAPI or PI may also be included. In a preferred embodiment, the kit comprises a probe. In one such embodiment the probe is a labeled probe. Alternatively, the probe is an indirectly-labeled probe. In this case, it is preferable to also include a secondary label. Staining buffers such as PBS plus 1% BSA can also be included in kit. In addition, in particular kits, crosslinking fixatives such as formaldehyde or paraformaldehyde can be included. In addition, a permeabilization agent such as the detergents Tween 20, Triton X-100, or saponin can also be part of a kit of the present invention.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Fluorescent In Situ Hybridization in Cells Fixed with a Polar Organic Solvent using Directly-Labeled Fluorescent Probes

Cells are washed in phosphate-buffered saline (PBS), then resuspended in 0.56% KCl at 2×10⁷ per ml or less, and incubated between about 25° C.-37° C. for 10-20 minutes to hypotonically swell cells. The optimal temperature and duration used for hypotonic swelling can depend on the cell type. Thus, the preferred method for swelling lymphoid or hematopoietic cells is 20 minutes at 37° C. The preferred method for swelling larger cells such as epithelial tumor cells is 10 minutes at 25° C. Cells are prefixed by adding 0.4 ml freshly made Carnoys (3:1 methanol:acetic acid) per ml of KCl, and pelleted using gentle centrifugation conditions (i.e., 600×g for 5 minutes). The supernatant is removed and cells are resuspended in fresh Carnoys at 10⁸ per ml or less. The hypotonic swelling step may be bypassed and the cells can be fixed directly in Carnoys, methanol or acetone. Fixed cells can be stored at 4° C. overnight (O.N.), or at −20° C. for long-term storage. At this point, the cells can be dropped on slides for conventional fluorescent in situ hybridization (FISH) or processed for FISH in suspension.

For FISH in suspension, 10⁷ or fewer cells are transferred to microfuge tubes and pelleted by centrifugation (i.e., 600×g for 5 minutes). The use of siliconized tubes minimizes cell loss but is not essential. The supernatant is removed and cells are resuspended in 1×SSC+0.05% BSA and rehydrated at 25° C. for 20 minutes to 1 hour. Cells can be left for several hours in 1×SSC without detriment or placed at 4° C. overnight. The cells are then pelleted by centrifugation (i.e., 5 minutes at 600×g), incubated with 2×SSC+0.05% BSA for 5-20 minutes then pelleted by centrifugation (i.e., 5 minutes at 600×g). The supernatant is carefully removed and cells are resuspended in hybridization buffer (2×SSC, 50% formamide, 10% dextran sulfate). 5-50 ng of labeled probe is added, and 10 μg blocking DNA (i.e., herring or salmon sperm DNA) is added if none is present in probe mixture. The final volume is adjusted with nuclease free water to 10-20 μl, depending on the volume of cell pellet.

The chromosomal DNA and probe are denatured together at 75° C. for 5 minutes or 80° C. for 45 seconds then hybridized at 37° C.-42° C. depending on the probe. For example, the X centromeric and Y α-satellite probes hybridize at either 37° C. or 42° C. The chromosome 13 and 21 locus specific probes and the chromosome 18 centromeric probe hybridize specifically at 37° C. If the directly labeled probe is commercially prepared and supplied pre-denatured, it is not usually adversely affected by denaturing it together with the chromosomal DNA. Hybridization proceeds for 30 minutes to overnight, depending on the probe. Following hybridization, cells are pelleted (i.e., 600×g) and hybridization buffer is removed. Cells are washed with 100-500 μl 2×SSC SSC plus 0.05% BSA, then pelleted as before. The supernatant is removed and cells are resuspended in 100-500 μl 2×SSC/50% formamide. Cells are incubated at 42° C. for 15-30 minutes to elute excess probe. An aliquot can be examined on a fluorescent microscope to determine if excess probe has been removed by mixing a 5 μl sample with an equal volume of antifade or antifade plus nuclear counterstain (i.e., DAPI or PI). Elution is continued if necessary. Signal is stable at 42° C. for several hours. Once excess probe has been eluted, cells are pelleted as before and washed in 2×SSC plus 0.05% BSA. At this stage the FISH signal can be evaluated by imaging flow cytometry, conventional flow cytometry, or fluorescence microscopy. This method has sufficient sensitivity for detection of single copy gene loci, such as Her-2/neu.

### Example 2

### In Situ Hybridization of Cells Fixed with a Polar Organic Solvent Using Indirectly-Labeled Probes

In situ hybridization may be performed using indirectly-labeled probes, i.e., biotin, digoxigenin or other ligand or hapten-labeled probes, followed by secondary label, i.e., avidin or anti-digoxigenin which contains a fluorescent or chromogenic substrate label. The cells are prepared as described in Example 1 above, for directly labeled fluorescent probes. However, hybridization and post-hybridization steps will vary accordingly. For example, if the indirectly-labeled probe is sensitive to heat denaturation (i.e., digoxigenin-labeled probes), the chromosomal DNA is denatured prior to adding the probe. The cells are resuspended in hybridization buffer as above, denatured at 75° C. for 5 minutes or 80° C. for 45 seconds, then the probe is added. If the probe is supplied pre-diluted in hybridization buffer, it may be necessary to pellet the cells and remove the excess hybridization buffer before adding the pre-diluted probe. Hybridization can proceed as described in Example 1 above. Following hybridization, excess probe is eluted and the cells are washed as described in Example 1 above. After the final 2×SSC wash step, cells are resuspended in the appropriate buffer (i.e., 4×SSC) for secondary detection with antidigoxigenin-conjugated fluorophore, streptavidin-conjugated flourophore or chromogenic substrate. This method is sufficiently sensitive to detect a single gene locus.

### Example 3

### Labeling of Cell Surface Antigens on Cells Fixed with a Polar Organic Solvent with Directly or Indirectly-Labeled Antibodies Followed by In Situ Hybridization in Suspension

Live cells are incubated with the desired antibodies to cell surface antigens prior to fixation according to standard immune-staining methods. For example, cells are incubated with either directly labeled fluorescent antibodies or indirectly-labeled antibodies (i.e., biotin conjugated antibody) in staining buffer such as PBS plus 1% BSA or fetal bovine serum (FBS). If antibodies are directly labeled, they must be labeled, e.g., with a fluorophore that withstands chemical fixation and heat denaturation, such as fluorescein isothiocyanate (FITC). Antibody labeled cells are then fixed with either methanol or acetone. Cells are rehydrated and hybridized as described in Example 1 above. Following hybridization and post-hybridization washes, cells are resuspended in staining buffer and indirectly-labeled antibodies are detected with secondary reagents (i.e., streptavidin conjugated fluorophone) according to standard detection methods.

### Example 4

### Labeling of Intracellular Antigens in Cells Fixed with a Polar Organic, Solvent Followed by In Situ Hybridization

Polar organic fixatives such as methanol, ethanol or acetone render the cells permeable to antibody molecules as well as to ISH probes, but problems arise from cell aggregation following fixation. The addition of a rehydration step prior to incubation with antibodies and ISH as disclosed herein, allows the cells to be resuspended so that they are suitable for analysis by flow cytometry.

Cells are fixed in 70-100% methanol, then pelleted and resuspended in staining buffer with a blocking protein, i.e., PBS+1% BSA or Hanks balanced salt solution (HBSS) plus 1% FBS. The cells are rehydrated in staining buffer for 10-20 minutes, then pelleted and resuspended in a smaller volume of staining buffer (i.e., 50-100 μl). Labeled antibody is added, and cells are incubated at room temperature for 30-60 minutes. Unlabeled antibody can also be used and detected subsequently with a secondary reagent. Preferably a directly labeled antibody is used to reduce nonspecific binding and the number of steps. Following antibody incubation the cells are washed in PBS+1% BSA, then resuspended in 1×SSC+0.05% BSA. The cells are then pelleted and resuspended in 2×SSC+0.05% BSA. The cells can then be used for ISH in suspension as described in Example 1 above. The intracellular antigen is preferably labeled with a fluorochrome that withstands heat denaturation such as FITC, rhodamine, the Cy dyes or the Alexa dyes. Protein based fluorochromes such as PE or PE-conjugates do not withstand heat denaturation but may be used as secondary reagents to detect intracellular antigens following ISH. Cell surface labeling can also be performed prior to fixation with methanol as described in Example 3 above. The antibody to the cell surface antigen should be labeled with a fluorochrome that is distinct from that used for intracellular antigen labeling. This method enables simultaneous analysis of cell surface antigens, intracellular antigens and chromosomal DNA.

### Example 5

### In Situ Hybridization of Aldehyde-Fixed/Heat Treated Cells with Directly-Labeled Fluorescent Probes

Cells are washed and resuspended in phosphate-buffered saline (PBS). The aldehyde fixative of choice (i.e., formaldehyde, paraformaldehyde, or glutaraldehyde) is added to a final concentration of 0.25-1%, depending on cell type. The cells are incubated at room temperature or 40° for 15 min to 1 hr, then washed with PBS. Fixed cells can be stored at 4° C. at this stage. Prior to ISH in suspension, the cells are pelleted and resuspended in 2×SSC. The cells are then heated to 65° C. for 1-4 hrs to reverse crosslinking of chromosomal DNA and nuclear proteins. The length of time required to reverse crosslinking depends on the type and percentage of aldehyde used for fixation, and the length of fixation time. In a preferred example, a volume of 0.25 ml of 2% paraformaldehyde is added to 0.75 ml of cells resuspended in PBS for a final concentration of 0.5% paraformaldehyde. The cells are allowed to fix for 30 minutes, then are washed 2× with PBS. The cells are pelleted and resuspended in 2×SSC, then subjected to a 65° C. heat treatment for 1 hr.

The cells can then be subjected to ISH in suspension as described previously. If it is desirable to perform ISH on slides, the cells can be resuspended in methanol and dropped on slides at this point for conventional ISH.

For ISH in suspension, the cells are transferred to microfuge tubes and pelleted by centrifugation (i.e., 600×g for 5 min). The supernatant is carefully removed and cells are resuspended in hybridization buffer (2×SSC, 50% formamide, 10% dextran sulfate). 5 ng of labeled probe is added, and 10 μg blocking DNA (i.e., herring or salmon sperm DNA) is added if none is present in probe mixture. Final volume is adjusted with nuclease free water to 10-20 μl, depending on cell pellet volume.

The chromosomal DNA and probe are denatured together at 75° for 5 min or 80° C. for 45 sec, then hybridized at 37°-42° C., depending on the probe used. For example, the fluorescently labeled X centromeric and Y α-satellite probes hybridize at either 37° or 42° C. The chromosome 13 and 21 locus specific probes and the chromosome 18 centromeric probe hybridize specifically at 37° C. If the directly labeled probe is commercially prepared and supplied pre-denatured, it is not usually adversely affected by denaturing it together with the chromosomal DNA. Hybridization proceeds from 30 min to overnight, depending on the probe. Following hybridization, cells are pelleted (i.e., 600×g) and hybridization buffer is removed. Cells are washed with 100-500 μl 2×SSC plus 0.05% BSA, then pelleted as before. The supernatant is removed and cells are resuspended in 100-500 μl 2×SSC/50% formamide. Cells are incubated at 42° C. for 15-30 min to elute excess probe. An aliquot can be examined on a fluorescence microscope to determine if excess probe has been removed by mixing a 5 μl sample with an equal volume of antifade, or antifade plus nuclear counterstain (i.e., DAPI or PI). Elution is continued if necessary. The signal has been established to be stable at 42° C. for several hours. If high background is observed, cells can be resuspended in 0.25×SSC and heated to 68° C. for 1-2 min to remove excess probe. Once excess probe has been eluted, cells are pelleted as before and washed in 2×SSC plus 0.05% BSA. At this stage, the FISH signal can be evaluated by imaging flow cytometry, conventional flow cytometry, or fluorescence microscopy. This method has sufficient sensitivity for detection of single copy gene loci, such as Her-2/neu.

### Example 6

### In Situ Hybridization of Aldehyde-Fixed/Heat Treated Cells Using Indirectly Labeled Probes

The cells are prepared in an identical manner as for directly labeled fluorescent probes. Hybridization and post hybridization steps vary according to the following: If the indirectly labeled probe is sensitive to heat denaturation (i.e., digoxigenin-labeled probes), the chromosomal DNA is denatured prior to adding the probe. The cells are resuspended in hybridization buffer as above, denatured at 75° C. for 5 min or 80° C. for 45 sec, then probe is added. If the probe is supplied pre-diluted in hybridization buffer, it is necessary to pellet the cells and remove the excess hybridization buffer before adding the pre-diluted probe. Hybridization proceeds as described above. Following hybridization, excess probe is eluted and cells washed as described above. After the final 2×SSC wash step, cells are resuspended in the appropriate buffer (i.e., 4×SSC) for secondary detection with anti-digoxigenin conjugated fluorophore, streptavidin conjugated flourophore, or chromogenic substrate. This method is also sufficiently sensitive to detect single gene loci.

### Example 7

### Labeling of Cell Surface Antigens on Aldehyde-Fixed/Heat Treated Cells with Directly or Indirectly Labeled Antibodies Followed by In Situ Hybridization

Viable cells are incubated with the desired antibodies to cell surface antigens prior to fixation according to standard immuno-staining methods. For example, cells are incubated with either directly labeled fluorescent antibodies or indirectly labeled antibodies (i.e., biotin conjugated antibody) in staining buffer such as PBS plus 1% BSA or fetal bovine serum (FBS). If antibodies are directly labeled, they must be labeled with a fluorophore that withstands chemical fixation and heat denaturation, such as fluorescein isothiocyanate (FITC), rhodamine, the Cy dyes, or the Alexa dyes. Phycoerythrin (PE) does not withstand heat denaturation, and can only be used as a secondary reagent following ISH. Antibody-labeled cells are then fixed in the aldehyde of choice, and processed as described in Example 5 above. Following hybridization and post-hybridization washes, cells are resuspended in staining buffer and indirectly labeled antibodies are detected with secondary reagents (i.e., streptavidin conjugated fluorophore) according to standard detection methods.

### Example 8

### Labeling of Cell Surface Antigens and/or Intracellular Proteins in Aldehyde-Fixed/Heat Treated Cells Followed by In Situ Hybridization

Viable cells are first incubated with antibodies to cell surface antigens if desired. The antibody should be directly labeled with a fluorescent dye that is capable of withstanding heat denaturation and chemical fixation. Cells are fixed with the aldehyde fixative of choice for 15-60 min, then permeabilized with a detergent for 30-60 min (i.e., Tween 20, Triton X-100, or saponin) to facilitate the entry of antibodies into the cell. A commercial fixation and permeabilization product such as Caltag Fix and Perm or Ortho Permeafix may be used. The cells are incubated with the desired antibody at room temp or 4° C. to label the intracellular protein either during the permeabilization step when using a saponin-based permeabilizing buffer, or following permeabilization when using Tween-20 or Triton X-100. A directly labeled fluorescent antibody gives optimal results with the least amount of nonspecific staining. The fluorescent label must be resistant to heat denaturation and chemical fixation. The intracellular antibody-antigen conjugate is then stabilized by the addition of methanol. Cells are fixed for 1 hr-O.N. at 4° C. in methanol, then pelleted and resuspended in 2×SSC. The cells are subsequently incubated at 65° C. for 1-4 hrs to reverse aldehyde-induced crosslinking prior to ISH in suspension. ISH in suspension is performed as described above. It is preferable to use distinct labels for the cell surface antigen and for the nucleic acid probe. If the intracellular protein is excluded from the nucleus, the intracellular label can be identical to the label used on the nucleic acid probe.

1. A method of identifying, detecting, or quantifying a specific cellular target molecule of interest in an intact cell in suspension, comprising:
(a) treating the intact cell that contains or is suspected to contain a specific cellular target molecule with a solution that comprises a polar organic solvent selected from a short-chain alcohol and acetone, wherein the treated cell becomes fixed;
(b) removing the fixed cell from the polar organic solvent of step (a);
(c) rehydrating the fixed cell in aqueous buffer to provide a rehydrated fixed intact cell;
(d) exposing the rehydrated fixed intact cell to a hybridization buffer;
(e) contacting the cell of step (d) with a probe able to hybridize to the specific cellular target molecule, wherein the specific cellular target molecule is a nucleic acid; and
(f) detecting the hybridized target molecule in the intact cell in suspension, wherein detecting comprises imaging flow cytometry.

2.-4. (canceled)

5. The method of claim 1, wherein the short-chained alcohol is selected from methanol and ethanol.

6. The method of claim 1, wherein the solution of step (a) further comprises acetic acid.

7. The method of claim 6, wherein the solution is 3:1 methanol:acetic acid.

8. The method of claim 1, wherein the intact cell is hypotonically swelled prior to step (a).

9. The method of claim 8, wherein the cell is swelled in a hypotonic salt solution.

10. The method of claim 1, wherein in step (b), the fixed cell is removed from the polar organic solvent by centrifugation.

11. The method of claim 1, wherein the probe is labeled or is contacted with a labeled binding partner.

12. The method of claim 11, wherein the label is selected from a luminescent label, a light absorbing label, a radioactive label, and a light scattering label.

13. (canceled)

14. The method of claim 1, wherein the nucleic acid encodes a protein.

15. The method of claim 14, wherein the protein is a cytokine or a hemoglobin chain.

16. (canceled)

17. The method of claim 1, wherein the nucleic acid is chromosomal DNA.

18.-27. (canceled)

28. A method of identifying, detecting, or quantifying a specific cellular target molecule in an intact cell in suspension, comprising:
(a) treating the intact cell that contains or is suspected to contain a specific cellular target molecule with an aldehyde fixative, wherein the treated intact cell becomes fixed;
(b) exposing the aldehyde-treated intact cell to a heat treatment;
(c) placing the cell of step (b) in a hybridization buffer;
(d) contacting the cell of step (c) with a probe able to bind or hybridize to the specific cellular target molecule, wherein the specific cellular target molecule is a nucleic acid; and
(e) detecting the hybridized target molecule in the intact cell in suspension, wherein detecting comprises imaging flow cytometry.

29.-30. (canceled)

31. The method of claim 28, wherein the aldehyde fixative is selected from formaldehyde, paraformaldehyde, and glutaraldehyde.

32. The method of claim 28, wherein the heat treatment of step (b) is conducted at a temperature between about 50° C.-70° C.

33. The method of claim 32, wherein the temperature is about 65° C.

34. The method of claim 28, wherein the heat treatment of step (b) is conducted between 30 min to 5 hours.

35. The method of claim 34, wherein the heat treatment is conducted between 1-4 hours.

36. The method of claim 28, wherein the probe is labeled or is contacted with a labeled binding partner.

37. The method of claim 36, wherein the label is selected from a luminescent label, a light absorbing label, a radioactive label, and a light scattering label.

38. (canceled)

39. The method of claim 28, wherein the nucleic acid encodes a protein.

40. The method of claim 39, wherein the protein is a cytokine or a hemoglobin chain.

41. (canceled)

42. The method of claim 28, wherein the nucleic acid is chromosomal DNA.

43.-71. (canceled)

72. A method of identifying, detecting or quantifying a specific cellular target molecule in an intact cell in suspension, comprising fixing an intact cell with a composition comprising a polar organic solvent selected from a short chain alcohol and acetone; rehydrating the fixed intact cell stepwise by subjecting said fixed intact cell to a plurality of rehydration steps with a plurality of aqueous buffers, such that said intact cell is prepared for in situ hybridization in suspension; and contacting the rehydrated fixed intact cell in suspension with a probe able to hybridize to the specific cellular target molecule, wherein the specific cellular target molecule is a nucleic acid, and wherein the probe that hybridizes to the specific cellular target molecule in the intact cell in suspension is further detected by imaging flow cytometry.

73. The method of claim 72, wherein the short-chain alcohol is selected from methanol and ethanol.

74. The method of claim 72, wherein the composition comprising a polar organic solvent further comprises acetic acid.

75. The method of claim 72, wherein the composition comprises 3:1 methanol:acetic acid.

76. The method according claim 72, wherein in the first rehydration step the fixed intact cell is subjected to a first aqueous buffer comprising 1×SSC.

77. The method of claim 76, wherein the first aqueous buffer further comprises a protein.

78. The method of claim 77, wherein the protein is bovine serum albumin or fetal bovine serum.

79. The method of claim 76, wherein in the second rehydration step the fixed intact cell is subjected to a second aqueous buffer comprising 2×SSC.

80. The method of claim 79, wherein the second aqueous buffer further comprises a protein.

81. The method of claim 80, wherein the protein is bovine serum albumin or fetal bovine serum.

82. The method of claim 72, wherein the intact cell is hypotonically swelled prior to fixation.

83. The method of claim 82, wherein the cell is swelled in a hypotonic salt solution.

84. The method of claim 72, wherein the probe is labeled or is contacted with a labeled binding partner.

85. The method of claim 84, wherein the label is selected from a luminescent label, a light absorbing label, a radioactive label, and a light scattering label.

86. The method of claim 72, wherein the nucleic acid molecule encodes a protein.

87. The method of claim 86, wherein the protein is a cytokine or a hemoglobin chain.

88. The method of claim 72, wherein the nucleic acid molecule is chromosomal DNA.

89. The method of claim 72, wherein the probe is further detected by microscopy.

90. A method for identifying, detecting, or quantifying a specific cellular target molecule in an intact cell in suspension, said method comprising:
(a) fixing an intact cell, which cell contains or is suspected of containing the specific cellular target molecule, with an aldehyde to provide an aldehyde-fixed cell;
(b) heating the aldehyde-fixed intact cell at about 50° C.-70° C. for a time ranging from about 30 minutes to about 5 hours;
(c) resuspending the cell of step (b) in a hybridization buffer;
(d) contacting the cell in suspension of step (c) with a probe that is capable of hybridizing to the specific cellular target molecule, wherein the specific cellular target molecule is a nucleic acid; and
(e) detecting the hybridized specific cellular target molecule in the intact cell in suspension wherein detecting comprises imaging flow cytometry.

91. The method of claim 90 wherein the aldehyde is formaldehyde, paraformaldehyde, or glutaraldehyde.

92. The method of claim 90 wherein the temperature is about 65° C.

93. The method of claim 90 wherein the probe is labeled or is contacted with a labeled binding partner.

94. The method of claim 93 wherein the label is selected from a luminescent label, a light absorbing label, a radioactive label, and a light scattering label.

95. The method of claim 90 wherein the nucleic acid encodes a protein.

96. The method of claim 95 wherein the protein is a cytokine or a hemoglobin chain.

97. The method of claim 90 wherein the nucleic acid molecule is chromosomal DNA.

98. The method of claim 90 wherein the probe is further detected by microscopy.
